(19) United States
(12) Patent Application Publication
(10) Pub. No.: US 2014/0296275 A1
(43) Pub. Date: Oct. 2, 2014

(54) Combinations of an Opioid/TLR4 Antagonist and an Alpha-2-Delta Ligand for Use in the Treatment of Pain

(71) Applicant: Annette Channa Toledano, North Miami, FL (US)

(72) Inventor: Annette Channa Toledano, North Miami, FL (US)

(21) Appl. No.: 13/851,773

(22) Filed: Mar. 27, 2013

(51) Int. Cl. A61K 31/485; A61K 31/195

(52) U.S. Cl. CPC A61K 31/485; A61K 31/195
USPC 514/282

(57) ABSTRACT

Disclosed are compositions for treatment of pain comprising, a first compound and a second compound, the first compound is an opioid antagonist that treats pain by blocking Toll-like receptor (TLR4) and the second compound is an alpha-2-delta ligand that enhances the pain treatment effect of the first compound. Examples of opioid antagonist include naltrexone, naloxone and nalmefene. Examples of an alpha-2-delta ligand include gabapentin and pregabalin, synergistic pharmaceutical compositions thereof, and their use in the treatment, prevention, and reversal of neuropathic pain.

### CROSS-REFERENCE TO RELATED APPLICATIONS

This application is a continuation-in-part of U.S. application Ser. No. 12/824,367 filed Jun. 28, 2010, which claims the benefit of U.S. Patent Provisional Application Ser. No. 61/343,489, filed on Apr. 29, 2010 and Provisional Application Ser. No. 61/395,772 entitled filed on May 17, 2010 the entire teachings of which are incorporated herein by reference.

### FIELD OF INVENTION

This invention relates to combinations of an opioid/TLR4 antagonist and an alpha-2-delta ligand, particularly those that exhibit a synergistic effect for the treatment, prevention and reversal of pain.

### BACKGROUND

It is well established in medical literature that treatments currently available for pain have limitations. Opioid drugs cause tolerance, dependence and side effects sufficiently serious to prompt recent action by the FDA to further restrict the drugs. Newly approved treatments, like the calcium channel alpha-2-delta ligands gabapentin and pregabalin and the serotonin and norepinephrine reuptake inhibitors milnacipran and duloxetine, require high doses to show nominal effectiveness, have a high dropout rate and carry many side effects.

This invention is a novel approach for the treatment of pain. It is directed to the treatment of neuropathic and nociceptive pain with an allodynic component. The two components of the combination are directed to reducing neuropathic pain and the allodynic component associated with nociceptive pain. Specific combinations of drugs and the dosage needed to create that effect is the subject of the instant invention.

In essence the instant invention recognizes that opioid receptor antagonists exert their action in a site other than the opioid receptors. That site is the immune system receptor TLR4 located on glia cells. The invention recognizes that the immune system going awry is the cause of neuropathic pain. Blocking TLR4s with an opioid receptor antagonist solves the perplexing problem of neuropathic pain. The instant invention is based on findings from a double-blind placebo-controlled clinical trial of 78 subjects treated with the opioid receptor naltrexone which proved the efficacy of this treatment for pain.

An alpha-2-delta ligand particularly Gabapentin and pregabalin or a pharmaceutically acceptable salt any thereof, enhances the pain relief action of the opioid/TLR4 antagonists particularly naltrexone. A specific synergistic dose range of the combination is herein presented.

In a dose finding study the combination of the opioid/TLR4 antagonist, naltrexone and the calcium channel alpha-2-delta ligands gabapentin and pregabalin, acted synergistically, whether administered separately, one after the other, or administered in combination.

Various p-opioid receptor ligands have been tested and were found to also possess action as agonists or antagonists of Toll-like receptor 4 (TLR4). Toll-like receptors, found in the glia, are a class of receptors that play a key role in the innate immune system. They recognize pathogen-associated molecular patterns (PAMPs) such as lipopolysaccharide (LPS) that are expressed on infectious agents, and mediate the production of cytokines necessary for the development of effective immunity. Opioid agonists such as morphine act as TLR4 agonists, while opioid antagonists such as naloxone and naltrexone were found to be TLR4 antagonists.

Activation of TLR4 by opioid agonists such as morphine leads to downstream release of inflammatory modulators including TNF-α and interleukin-1. Constant low-level release of these modulators is thought to reduce the efficacy of opioid drug treatment with time and to be involved in both the development of tolerance to opioid analgesic drugs and in the emergence of side effects such as hyperalgesia and allodynia which can become problems following extended use of opioid drugs.

Accordingly, the instant invention relates to p-opioid receptor ligand as ligands of TLR4 as well and contemplates that allodynia is caused by activation of TLR4. Blockage of TLR4 accordingly will eliminate allodynia.

Several opioid antagonist drugs were found to act as antagonists for TLR4, including naloxone, naltrexone and nalmefene. However it was found that not only the “normal” (−) enantiomers, but also the “unnatural” (+) enantiomers of these drugs acted as TLR4 antagonists. The unnatural enantiomers of the opioid antagonists, (+)-naltrexone and (+)-naloxone, dextro-naltrexone and dextro-naloxone, have been discovered to act as selective antagonists of TLR4. Since (+)-naloxone and (+)-naltrexone lack affinity for opioid receptors, they do not block the effects of opioid analgesic drugs, and so can be used to counteract the TLR4-mediated side effects of opioid agonists without affecting analgesia. (+)-Naloxone was also found to be neuroprotective, and both (+)-naloxone and (+)-naltrexone are effective in their own right at treating symptoms of neuropathic pain in animal models.

The best known opioid receptor antagonists are naltrexone, naloxone and nalmefene. Naltrexone is an opioid receptor antagonist used primarily in the management of alcohol dependence and opioid dependence. A dose of 50-300 mg once daily is recommended for most patients. Naloxone is an opioid inverse agonist: it is a drug used to counter the effects of opiate overdose.

Low dose naltrexone describes the off label use of naltrexone at doses less than 15 mg per day for indications other than chemical dependency or intoxication.

It has been suggested in the literature that low dose naltrexone exerts the opposite effect of naltrexone in full dose. While the full dose naltrexone blocks the opiate system, the low dose naltrexone promotes the production of endorphins by the mechanism of up regulation caused by partial opiate receptor blockage. The beneficial effect of naltrexone was attributed to the increase in endorphins. The beneficial effect of low dose naltrexone can be further explained by its antagonism of TLR4.

Other opioid receptor antagonists used in clinical or scientific practice which also can be used for the treatment of pain include but are not limited to the following: naloxone, nalmefene, norbinaltorphimine, nalorphine, methylnaltrexone, samidorphan, cyprodime, naltrindole, amentoflavone, naltriben, norbinaltorphimine, and the naltrexone metabolite 6-β-naltrexol.

Our understanding of pathological pain has primarily revolved around neuronal mechanisms. However, neighboring glia, were TLL4 reside, including astrocytes and microglia; have recently been recognized as powerful modulators of pain.

Studies show that TLRs can be activated not only by well-known “non-self” molecular signals but also by endogenous signals (IL-1β, TNFα, IL-6 and NO) produced during chronic neuropathic pain states. Fibronectin, an endogenous TLR4 ligand that is produced in response to tissue injury, leads to an up regulation of the purinoceptor P2X4, which is expressed exclusively on microglia.

Voltage-dependent calcium channels alpha-2-delta-1 and alpha-2-delta-2 subunits are the binding site of the two anticonvulsant drugs, gabapentin (Neurontin) and pregabalin (Lyrica), that also find use in treating chronic neuropathic pain.

Gabapentin (Neurontin) is a pharmaceutical drug, specifically a GABA analog. It was originally developed for the treatment of epilepsy, and currently is also used to relieve neuropathic pain. Gabapentin provides significant pain relief in about a third of people who take it for fibromyalgia or chronic neuropathic pain.

Pregabalin is an anticonvulsant drug used for neuropathic pain. Recent studies have shown that pregabalin is effective at treating chronic pain in disorders such as fibromyalgia

Gabapentin and Pregabalin enhance the pain treatment effect of naltrexone by treating pain via another pathway, by binding Voltage-dependent calcium channels alpha-2-delta.

Allodynia is a clinical feature of many painful conditions, such as back pain, chronic pain, neuropathic pain, diabetic neuropathic pain, trigeminal neuralgia pain, phantom limb pain, complex regional pain syndrome pain, acute herpetic pain, post herpetic pain, causalgia pain, idiopathic pain, inflammatory pain, cancer pain, postoperative pain, fibromyalgia pain, headache pain, migraine pain, allodynia pain, vulvodynia pain, interstitial cystitis pain, irritable bowel syndrome (IBS), arthritic joint pain and tendinitis. It becomes apparent that allodynia plays a role in every kind of pain.

The instant invention offers a new explanation for the occurrence of allodynia, or “memory pain”, connecting the dots of existing knowledge from animal model studies along with the vast information gleaned from the instant invention clinical trials, it is now evident that allodynia is caused by abnormal endogenous activation of TLR4 that in turn trigger a pro-inflammatory cascade. The instant invention's clinical trial for back pain verified that the pain is interrupted by the opioid/TLR4 antagonist naltrexone. Additionally, TLR4 antagonism can play a role in improving nociceptive pain by affecting the allodynic component of nociceptive pain.

Based upon this, the instant invention first teaches the use of an opioid/TLR4 antagonist, particularly naltrexone for its antagonism of the TLR4 and blocking release of inflammatory modulators. Secondly, the invention teaches use of an alpha-2-delta ligand, particularly Gabapentin or Pregabalin, for their action on neuropathic pain. The invention teaches that the combination is synergistic as far as the effect on pain.

The invention contemplates several forms of opioid antagonist selected from a group consisting of naltrexone, naloxone, nalmefene, norbinaltorphimine, nalorphine, methylnaltrexone, samidorphan, cyprodime, naltrindole, amentoflavone, naltriben, norbinaltorphimine, and metabolite 6-8-naltrexol and metabolites and pro drugs thereof, including all enantiomeric and epimeric forms as well as the appropriate mixtures thereof, or pharmaceutically acceptable salts or solvates of any thereof.

The invention contemplates two forms of alpha-2-delta ligand selected from Gabapentin or Pregabalin.

### SUMMARY OF INVENTION

The instant invention is a synergistic combination product comprising a first compound and a second compound, where the first compound is an opioid antagonist that treats neuropathic pain by blocking receptor TLR4 and the second compound is an alpha-2-delta ligand that treats neuropathic pain as well, it enhances the pain treatment effect of the first compound. Another invention embodiment is a method for the treatment, prevention, and reversal of pain, particularly neuropathic pain.

### DESCRIPTION OF EMBODIMENTS

This invention provides a combination, comprising an opioid/TLR4 antagonist, and pharmaceutically acceptable salts or solvates of any thereof, and an alpha-2-delta ligand, and pharmaceutically acceptable salts or solvates of any thereof.

Another invention embodiment is a combination, comprising an opioid antagonist and an alpha-2-delta ligand. The opioid/TLR4 antagonist is selected from a group consisting of naltrexone, norbinaltorphimine, nalmefene, naloxone, nalorphine, methylnaltrexone, samidorphan, cyprodime, naltrindole, amentoflavone, naltriben, norbinaltorphimine, 6-β-naltrexol and metabolites thereof, including all enantiomeric and epimeric forms as well as the appropriate mixtures thereof, as well as pro drugs or metabolites thereof or pharmaceutically acceptable salts or solvates of any thereof.

Another invention embodiment is a combination, comprising an opioid antagonist and an alpha-2-delta ligand. Wherein an alpha-2-delta ligand inhibitor is selected from Gabapentin or Pregabalin or pharmaceutically acceptable salts or solvates of any thereof.

Another invention embodiment is a combination, comprising an opioid antagonist and an alpha-2-delta ligand, the opioid antagonist/TLR4 is naltrexone as well as pro drugs and all enantiomeric and epimeric forms, specifically, (+)-naltrexone (dextro-naltrexone), as well as the appropriate mixtures thereof, or pharmaceutically acceptable salts or solvates of any thereof.

Another invention embodiment is a combination, comprising an opioid antagonist and an alpha-2-delta ligand, the opioid antagonist/TLR4 is naltrexone in a sustained release formulation, as well as pro drugs thereof or any enantiomeric and epimeric forms thereof, as well as the appropriate mixtures thereof, or pharmaceutically acceptable salts or solvates of any thereof.

Another invention embodiment is a combination, comprising an opioid antagonist and an alpha-2-delta ligand, the opioid antagonist/TLR4 is (+)-naltrexone (dextro-naltrexone), as well as pro drugs thereof or any enantiomeric and epimeric forms thereof, as well as the appropriate mixtures thereof, or pharmaceutically acceptable salts or solvates of any thereof.

Another invention embodiment is a combination, comprising naltrexone, or a pharmaceutically acceptable salt or solvate thereof, and Gabapentin or Pregabalin, or a pharmaceutically acceptable salt or solvate thereof.

Another invention embodiment is a combination, comprising naltrexone and Gabapentin in a weight to weight combination range which corresponds to a synergistic combination range of the order of 1:50-1:125 parts by weight.

Another invention embodiment is a combination, comprising naltrexone and Pregabalin in a weight to weight combination range which corresponds to a synergistic combination range of the order of 1:30-1:50 parts by weight.

Another invention embodiment is a combination, comprising the dose range of naltrexone, or a pharmaceutically acceptable salt or solvate thereof, is about 0.004 mg/kg-0.71 mg/kg per day.

Another invention embodiment is a combination, comprising the dose range of Gabapentin, or a pharmaceutically acceptable salt or solvate thereof, is about 1.3 mg/kg-26 mg/kg per day.

Another invention embodiment is a combination, comprising the dose range of Pregabalin, or a pharmaceutically acceptable salt or solvate thereof, is about 2 mg/kg-4 mg/kg per day.

Another invention embodiment is a combination, comprising the human dose range of naltrexone, or a pharmaceutically acceptable salt or solvate thereof, is 0.25 mg-50 mg per day.

Another invention embodiment is a combination, comprising the human dose range of naltrexone, or a pharmaceutically acceptable salt or solvate thereof, is 0.25 mg-25 mg per day.

Another invention embodiment is a combination, comprising the human dose range of naltrexone, or a pharmaceutically acceptable salt or solvate thereof, is in “low” dose of 0.25 mg-15 mg per day.

Another invention embodiment is a combination, comprising a human dose range of Gabapentin, or a pharmaceutically acceptable salt or solvate thereof, is 100 mg-1800 mg per day.

Another invention embodiment is a combination, comprising a human dose range of Pregabalin, or a pharmaceutically acceptable salt or solvate thereof, is 150 mg-300 mg per day.

Another invention embodiment is a combination, comprising the human dose range of naltrexone, or a pharmaceutically acceptable salt or solvate thereof, is 0.25 mg-50 mg per day, and the human the dose range of Gabapentin, or a pharmaceutically acceptable salt or solvate thereof, is 100 mg-1800 mg, wherein said composition is formulated into a single fixed combination dosage form.

Another invention embodiment is a combination, comprising the human dose range of naltrexone, or a pharmaceutically acceptable salt or solvate thereof, is 0.25 mg-50 mg per day, and the human the dose range of pregabalin, or a pharmaceutically acceptable salt or solvate thereof, is 50 mg-300 mg, wherein said composition is formulated into a single fixed combination dosage form.

Another invention embodiment comprising the composition is administered once, twice, three or four times through the day.

Another invention embodiment comprising the therapeutically effective dose of the pharmaceutical composition is administered systemically by such routes including but are not limited to mucosal, nasal, oral, parenteral, gastrointestinal, topical or sublingual routes.

Another invention embodiment comprising, said combination is in a single dosage form, and said single dosage form is in the form of tablets, lozenges, troches, hard candies, liquid, powders, sprays, creams, salves and suppositories.

Another invention embodiment the pharmaceutical composition is used for the treatment, prevention and reversal of neuropathic pain, back pain, chronic pain, diabetic neuropathic pain, trigeminal neuralgia pain, phantom limb pain, complex regional pain syndrome pain, acute herpetic pain, post herpetic pain, causalgia pain, idiopathic pain, inflammatory pain, cancer pain, postoperative pain, fibromyalgia pain, headache pain, migraine pain, allodynia pain, vulvodynia pain, interstitial cystitis pain, irritable bowel syndrome (IBS), arthritic joint pain and tendinitis.

Another invention embodiment is a method of treating neuropathic and nociceptive pain with an allodynic component and migraine in a mammal in need thereof, comprising administering to the mammal a therapeutically effective amount of a combination comprising an opioid/TLR4 antagonist and an alpha-2-delta ligand, or pharmaceutically acceptable salts or solvates of any thereof.

Another invention embodiment is a method of treating neuropathic and nociceptive pain with an allodynic component and migraine in a mammal in need thereof, comprising administering to the mammal a therapeutically effective amount of a combination comprising naltrexone and Gabapentin or Pregabalin, or pharmaceutically acceptable salts or solvates of any thereof.

Another invention embodiment, the combination of naltrexone, or a pharmaceutically acceptable salt or solvate thereof, and Gabapentin or Pregabalin, or a pharmaceutically acceptable salt solvate thereof, may optionally be administered with one or more other pharmacologically active agents. Appropriate optional agents include: aspirin, ibuprofen, naproxen, naprosyn, diclofenac, ketoprofen, tolmetin, sulindac, mefanamic acid, meclofenamic acid, diflunisal, flufenisal, piroxicam, sudoxicam, isoxicam, celecoxib, fofecoxib, flosulide, meloxicam, 6-methoxy-2-naphthylacetic acid, nabumetone, nimesulide, steroidal anti-inflammatory drugs, tricyclic antidepressants (TCAs), selective serotonin reuptake inhibitors (SSRIs), serotonin-norepinephrine reuptake inhibitors (SSRIs), anticonvulsants, muscle relaxants, drugs with NMDA antagonist properties, tetrahydrocannabinol derivatives, antitussive, expectorants, decongestants, or antihistamines.

Another invention embodiment for non-human animal administration the term “pharmaceutical” as used herein may be replaced by “veterinary”.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

### Pharmaceutical Composition

Naltrexone and Gabapentin or Pregabalin were evaluated alone and in combination on a human subject with the purpose of finding whether or not a combination of the two compounds offers a synergistic advantage for the pain treatment effect comparing the amounts used weight to weight.

The components of the combination were administered to a subject as follows: the naltrexone dose administered alone was 4.5 mg, and the Gabapentin and pregabalin dose administered alone was 1800 mg and 300 mg respectively. The dose of the naltrexone/Gabapentin combination was 2.25 mg/300 respectively and the naltrexone/pregabalin combination was 2.25 mg/150 respectively, the pain treatment effect was evaluated one hour post-dose.

To determine synergy, the amounts of naltrexone and Gabapentin or pregabalin administered alone were compared to the combination combined amounts. For proper weight to weight (W/W) comparison between naltrexone and Gabapentin or pregabalin an adjustment for the higher potency of naltrexone was made based on the dose of each compound given by itself. Naltrexone is 200 times more potent than gabapentin (200/4.5=200). Naltrexone and Gabapentin were administered at fixed dose ratios of 1:50-1:125 to a human subject afflicted with neuropathic back pain. The 1:125 combinations represent a 2-fold lower dose of naltrexone and 6-fold lower dose of Gabapentin.

Table 1 illustrates the naltrexone/Gabapentin ratio that exhibit weight to weight (W/W) synergy in a human subject. The 1:50 combinations represent a 2-fold lower dose of naltrexone and 18 fold lower dose of Gabapentin. The 1:125 combinations represent a 2-fold lower dose of naltrexone and 6-fold lower dose of Gabapentin.

TABLE 1
Naltrexone/Gabapentin Ratios
And Weight to Weight (W/W) Synergy

| | | | | | Total dose | |
|---|---|---|---|---|---|---|
| | | | Naltrexone | | Naltrexone | |
| | | | Potency | | + | |
| | | Gaba- | Adjust- | % | Adjusted | |
| | Naltrexone | pentin | ment | reversal | Gabapentin | Inter- |
| Ratio | mg | mg | (×200) | of pain | mg | action |
| 4.5:0 | 4.50 | — | 900.00 | 100 | 900 | — |
| 0:900 | — | 1800 | — | 50 | 1800 | — |
| 1:50 | 2.25 | 100 | 450.00 | 100 | 100 + 450 = | Synergy |
| | | | | | 550 | |
| 1:125 | 2.25 | 300 | 450.00 | 100 | 300 + 450 = | Synergy |
| | | | | | 850 | |

Table 2 illustrates the naltrexone/pregabalin ratio that exhibit weight to weight (W/W) synergy in a human subject. The 1:30 combinations represent a 2-fold lower dose of naltrexone and 4 fold lower dose of pregabalin when administered together. The 1:50 combinations represent a 2-fold lower dose of naltrexone and 2.4 fold lower dose of Gabapentin when administered together.

TABLE 2
Naltrexone/pregabalin Ratios
And Weight to Weight (W/W) Synergy

| | | | | | Total dose | |
|---|---|---|---|---|---|---|
| | | | Naltrexone | | Naltrexone | |
| | | | Potency | | + | |
| | Nal- | pre- | Adjust- | % | Adjusted | |
| | trexone | gabalin | ment | reversal | pregabalin | Inter- |
| Ratio | mg | mg | (×75) | of pain | mg | action |
| 4.5:0 | 4.50 | — | 300.00 | 100 | 300 | — |
| 0:900 | — | 300 | — | 50 | 300 | — |
| 1:30 | 2.25 | 75 | 150.00 | 100 | 150 + 75 = | Synergy |
| | | | | | 225 | |
| 1:40 | 2.25 | 100 | 150.00 | 100 | 150 + 100 = | Synergy |
| | | | | | 250 | |
| 1:50 | 2.25 | 125 | 150.00 | 100 | 150 + 125 = | Synergy |
| | | | | | 275 | |

That which is claimed is:

1. A composition for treatment of pain in a mammal comprising a synergistic ratio of (a) an opioid/TLR4 antagonist, or pharmaceutically acceptable salts or solvates thereof and (b) an alpha-2-delta ligand, or pharmaceutically acceptable salts or solvates thereof.

2. A composition comprising the formulation of claim 1, wherein the opioid/TLR4 antagonist is selected from a group consisting of naltrexone, norbinaltorphimine, nalmefene, naloxone, nalorphine, methylnaltrexone, samidorphan, cyprodime, naltrindole, amentoflavone, naltriben, norbinaltorphimine, 6-alpha-naltrexol, 6-beta-naltrexol metabolites and pro drugs thereof, including all enantiomeric and epimeric forms as well as the appropriate mixtures thereof, or pharmaceutically acceptable salts or solvates of any thereof.

3. A composition comprising the formulation of claim 1, wherein the opioid/TLR4 antagonist is naltrexone as well as pro drugs thereof or any enantiomeric and epimeric forms thereof, as well as the appropriate mixtures thereof, or pharmaceutically acceptable salts or solvates of any thereof.

4. A composition comprising the formulation of claim 3, wherein the opioid/TLR4 antagonist is naltrexone in a sustained release formulation, as well as metabolites and pro drugs thereof or any enantiomeric and epimeric forms thereof, as well as the appropriate mixtures thereof, or pharmaceutically acceptable salts or solvates of any thereof.

5. A composition comprising the formulation of claim 3, wherein the opioid/TLR4 antagonist is (+)-naltrexone (dextro-naltrexone), as well as appropriate mixtures thereof, as well as metabolites or pro drugs thereof, or pharmaceutically acceptable salts or solvates thereof.

6. A composition comprising the formulation of claim 1, wherein the alpha-2-delta ligand is selected from Gabapentin or Pregabalin or pharmaceutically acceptable salts or solvates of any thereof.

7. A composition comprising the formulation of claim 1, wherein the alpha-2-delta ligand inhibitor is Gabapentin, or pharmaceutically acceptable salts or solvates thereof.

8. A composition comprising the formulation of claim 1, wherein the alpha-2-delta ligand inhibitor is pregabalin, or pharmaceutically acceptable salts or solvates thereof.

9. A composition according to claim 1, wherein the opioid/TLR4 antagonist is naltrexone, or pharmaceutically acceptable salts or solvates thereof, in a therapeutically effective amount and the alpha-2-delta inhibitor is Gabapentin or Pregabalin, or pharmaceutically acceptable salts or solvates thereof, in a therapeutically effective amount.

10. A composition according to claim 1, wherein the opioid/TLR4 antagonist is dextro naltrexone, or pharmaceutically acceptable salts or solvates thereof, in a therapeutically effective amount and the alpha-2-delta inhibitor is Gabapentin or Pregabalin, or pharmaceutically acceptable salts or solvates thereof, in a therapeutically effective amount.

11. A composition according to claim 9, wherein naltrexone and alpha-2-delta ligand, or pharmaceutically acceptable salts or solvates of any thereof, are in a weight to weight combination range which corresponds to a synergistic combination of 1:30-1:125 parts by weight.

12. A composition according to claim 10, wherein the dose range of naltrexone, or pharmaceutically acceptable salts or solvates thereof, is about 0.004 mg/kg-0.71 mg/kg.

13. A composition according to claim 10, wherein the human dose range of naltrexone is 0.25 mg-50 mg per day.

14. A composition according to claim 10, wherein the human dose range of naltrexone is 0.25 mg-25 mg per day.

15. A composition according to claim 10, wherein the human dose range of naltrexone is 0.25 mg-15 mg per day.

16. A composition according to claim 10, wherein said composition is formulated into a single fixed combination dosage form and wherein, the composition is administered once, twice, three or four times through the day.

17. A composition of claim 10, wherein the therapeutically effective dose of the pharmaceutical composition is administered systemically, including but are not limited to mucosal, nasal, oral, parenteral, gastrointestinal, topical or sublingual routes.

18. A composition, according to claim 10, wherein said combination is in a single dosage form, and wherein, said single dosage form is in the form of tablets, lozenges, troches, hard candies, liquid, powders, sprays, creams, salves and suppositories.

19. A composition, according to claim 1 for treating, preventing and reversing pain.

20. A method of treating neuropathic pain, nociceptive pain with an allodynic component, migraine, trigeminal neuralgia, vulvodynia, irritable bowel syndrome, post herpetic neuralgia, or diabetic neuropathy in a mammal in need thereof, comprising administering to the mammal in a therapeutically effective amount of a combination according to claim 10.
